SEQUENCE LISTING (11 sequences)

SEQ ID NO: 1 - DNA, 22 bp, Artificial sequence
  Synthetic primer
ggcccggtgagagactccacac

SEQ ID NO: 2 - DNA, 30 bp, Artificial sequence
  Synthetic primer
ccaggagatgtaactctaatccaggtttgc

SEQ ID NO: 3 - DNA, 20 bp, Artificial sequence
  Synthetic primer
gcaagaagaagatcgccaag

SEQ ID NO: 4 - DNA, 20 bp, Artificial sequence
  Synthetic primer
tccaaggggatatccacaga

SEQ ID NO: 5 - DNA, 20 bp, Artificial sequence
  Synthetic primer
tggctacaatggggaaactg

SEQ ID NO: 6 - DNA, 19 bp, Artificial sequence
  Synthetic primer
ctgctgcctcttccatttg

SEQ ID NO: 7 - DNA, 20 bp, Artificial sequence
  Synthetic primer
gatcaatctccccatccaga

SEQ ID NO: 8 - DNA, 20 bp, Artificial sequence
  Synthetic primer
tggcagagggtctctgtctt

SEQ ID NO: 9 - DNA, 16 bp, Artificial sequence
  Synthetic primer
gccggcccaggtacca

SEQ ID NO: 10 - DNA, 17 bp, Artificial sequence
  Synthetic primer
cagcgagctcccttgca

SEQ ID NO: 11 - DNA, 14 bp, Artificial sequence
  Synthetic probe
cagtgcgcaccccg

(19) United States
(12) Patent Application Publication
(10) Pub. No.: US 2020/0297696 A1
(43) Pub. Date: Sep. 24, 2020

(54) P38 INHIBITORS FOR THE TREATMENT OF FSHD

(71) Applicants: Saint Louis University, St. Louis, MO (US); Fred Hutchinson Cancer Research Center, Seattle, WA (US)

(72) Inventors: Francis M. SVERDRUP, Lake Saint Louis, MO (US); Stephen J. TAPSCOTT, Seattle, WA (US); Jonathan OLIVA, Webster Groves, MO (US); Amy E. CAMPBELL, Seattle, WA (US); Marvin J. MEYERS, Wentzville, MO (US)

(73) Assignees: Saint Louis University, St. Louis, MO (US); Fred Hutchinson Cancer Research Center, Seattle, WA (US)

(21) Appl. No.: 16/766,030

(22) PCT Filed: Nov. 16, 2018
(86) PCT No.: PCT/US2018/061454

§ 371 Date: May 21, 2020

Related U.S. Application Data
(60) Provisional application No. 62/589,225, filed on Nov. 21, 2017.
(60) Provisional application No. 62/726,696, filed on Sep. 4, 2018.

(51) Int. Cl. A61K 31/415; A61K 9/00; A61K 31/495; A61K 31/506; A61K 31/437; A61K 31/519; A61K 31/44; A61K 31/5513; A61K 31/4412; A61K 31/4439; A61K 31/496; A61K 31/5025; A61P 21/00

(52) U.S. Cl. CPC A61K 31/415; A61K 9/0019; A61K 9/0053; A61K 31/495; A61K 31/506; A61K 31/437; A61P 21/00; A61K 31/44; A61K 31/5513; A61K 31/4412; A61K 31/4439; A61K 31/496; A61K 31/5025; A61K 31/519

(57) ABSTRACT

The present disclosure provides methods of treating a patient comprising administering a p38 inhibitor for the treatment of FSHD. In some embodiments, the present methods comprise using one or more p38 inhibitors as a therapeutic agent for the treatment of FSHD patients including patients who are being treated with one or more palliative treatments such as therapy and/or agents which lead to increased muscle mass.

### CROSS-REFERENCE TO RELATED APPLICATIONS

This application claims benefit of priority to U.S. Provisional Application Ser. No. 62/589,225, filed Nov. 21, 2017, and U.S. Provisional Application Ser. No. 62/726,696, filed Sep. 4, 2018, the entire contents of both applications being hereby incorporated by reference.

### GOVERNMENT SUPPORT CLAUSE

This invention was made with government support under Grant No. NS069539 awarded by the National Institutes of Health. The government has certain rights in the invention.

### BACKGROUND

### I. Field

The present disclosure relates to the fields of pharmaceuticals, medicine and cell biology. More specifically, it relates to pharmaceutical agents (compounds) which are useful in the treatment of facioscapulohumeral muscular dystrophy (FSHD).

### II. Description of Related Art

Facioscapulohumeral dystrophy (FSHD) is a prevalent muscular dystrophy affecting over 800,000 individuals worldwide. The disease typically presents in young adults as facial and upper extremity weakness and progresses to involve nearly all skeletal muscle groups [Tawil, et al., 2014]. FSHD is caused by the mis-expression of the double homeobox 4 (DUX4) transcription factor in skeletal muscle. Because of its causative role in FSHD, suppressing DUX4 expression is a primary therapeutic approach for halting disease progression. However, the mechanisms responsible for DUX4 expression are poorly understood and limited drug targets have been identified. Consequently, there is currently no treatment available for FSHD and few clinical trials of promising therapies are ongoing. Therefore, there remains a need to identify new therapeutic agents that may be used in the treatment of FSHD.

### SUMMARY

In accordance with the present disclosure, there is provided a method of using p38 inhibitors to treat facioscapulohumeral muscular dystrophy (FSHD). Without wishing to be bound by any theory, it is believed that the p38 inhibitors may modulate the expression of DUX4 and thus exert their therapeutic activity.

In some aspects, the present disclosure provides methods of treating a patient with facioscapulohumeral muscular dystrophy (FSHD) comprising administering to the patient a therapeutically effective amount of an inhibitor of p38. In some embodiments, the inhibitor of p38 is an inhibitor of p38α and/or p38β such as a selective inhibitor of p38α or a selective inhibitor of p38β. In some embodiments, the inhibitor of p38 modulates the expression of DUX4. The inhibitor of p38 may not inhibit the MK2 pathway. In other embodiments, the inhibitor of p38 does not inhibit either p38δ or p38γ. In some embodiments, the inhibitor of p38 may be selected from acumapimod, ARRY-371797, pexmetinib, AS1940477, BMS-582949, dilmapimod, dorimapimod, losmapimod, LY2228820, LY3007113, pamapimod, PH-797804, SB202190, SB203580, TAK-715, talmapimod, VX-702, and VX-745. In an exemplary embodiment, the inhibitor of p38 is losmapimod.

In some embodiments, the FSHD has been diagnosed. In other embodiments, the FSHD has not been diagnosed. In some embodiments, the FSHD is adult-onset FSHD. In other embodiments, the FSHD is infantile-onset FSHD. In some embodiments, the FSHD is Type 1 FSHD. In other embodiments, the FSHD is Type 2 FSHD. In some embodiments, the patient is a mammal such as a human, or a non-human mammal such as a rat, a mouse, a rabbit, a dog or a cat. In some embodiments, the patient exhibits one or more symptoms of FSHD such as facial muscle weakness, shoulder weakness, hearing loss, abnormal heart rhythm, unequal weakening of muscles in the upper body, loss of strength in the abdominal muscles, or foot drop. In some embodiments, the symptom of FSHD is facial muscle weakness. In some embodiments, the unequal weakening of muscles in the upper body is unequal weakening of muscles in the biceps, triceps, deltoids, or lower arm muscles.

In some embodiments, the methods further comprise administering a second therapy for FSHD. In some embodiments, the second therapy is administered before the inhibitor of p38. In other embodiments, the second therapy is administered concurrently with the inhibitor of p38. In other embodiments, the second therapy is administered after the inhibitor of p38. In some embodiments, the second therapy is a BET inhibitor such as I-BET762, I-BET726, I-BET151, RVX-208, CPI-203, CPI-232, CPI-0610, (+) JQ1, OTX-015, GW-841819X, BET-BAY-022, SRX-2523, or ABBV-075. In other embodiments, the second therapy is a 0-2 adrenergic receptor agonist such as bitolterol, fenoterol, isoprenaline, levosalbutamol, orciprenaline, pirbuterol, procaterol, ritodrine, salbutamol, bambuterol, formoterol, arformoterol, clenbuterol, salmeterol, abediterol, indacaterol, or olodaterol. In other embodiments, the second therapy comprises the BET inhibitor and the β-2 adrenergic receptor agonist such as when the BET inhibitor is I-BET762, I-BET726, I-BET151, RVX-208, CPI-203, CPI-232, CPI-0610, (+) JQ1, OTX-015, GW-841819X, BET-BAY-022, SRX-2523, or ABBV-075 and the β-2 adrenergic receptor agonist is bitolterol, fenoterol, isoprenaline, levosalbutamol, orciprenaline, pirbuterol, procaterol, ritodrine, salbutamol, bambuterol, formoterol, arformoterol, clenbuterol, salmeterol, abediterol, indacaterol, or olodaterol. In some embodiments, the second therapy is a therapy to increase muscle mass, physical therapy, or occupational therapy. In some embodiments, the second therapy is a therapy which improves the quality of life. In some embodiments, the second therapy is scapular fusion or scapular bracing. In other embodiments, the second therapy is an anti-inflammatory compound such as an NSAID or a glucocorticoid receptor modulator (glucocorticoid).

In some embodiments, the compound is administered systemically such as via injection or oral administration. In some embodiments, the inhibitor of p38 is administered once. In other embodiments, the inhibitor of p38 is administered two or more times.

In some aspects, the present disclosure provides pharmaceutical compositions formulated for the treatment of facioscapulohumeral muscular dystrophy (FSHD) comprising a p38 inhibitor and an excipient. In some embodiments, the pharmaceutical compositions are formulated for administration orally or via injection. In other embodiments, the pharmaceutical compositions are formulated for administration via injection such as via intravenous, subcutaneous, or intramuscular injection. The pharmaceutical composition may be formulated as a unit dose.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula does not mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1I show p38 inhibitors reduce DUX4 target MBD3L2 expression at concentrations that do not affect differentiation. Differentiating cultures of FSHD2 patient-derived muscle cells were treated with varying concentration of p38 inhibitors, as indicated, for 40 hours and the cultures analyzed for DUX4 target MBD3L2 or differentiation markers MYOG and MYH2 RNA levels. Data is expressed as relative expression with the expression in absence of inhibitors set to one.

FIG. 2 shows the p38 inhibitor SB203580 inhibits DUX4 expression. FSHD myoblasts were induced to differentiate for 48 hours. The cultures were then treated with the p38 inhibitor SB203580 (10 uM) or DMSO as a control for the last 16, 20 or 24 hours of the 48-hour differentiation period. Endogenous DUX4 mRNA expression, as well as DUX4 target LEUTX, was suppressed when SB203580 was added while the differentiation marker Desmin was unaffected. RPL27 was used as a control gene and was not affected by treatment.

FIG. 3 shows the p38 inhibitor PH-797804 reduces DUX4 and DUX4 target gene expression at concentrations that do not affect differentiation. Differentiating cultures of FSHD1 (54-2) and FSHD2 (MB200) patient-derived muscle cells were treated with varying concentrations of PH-797804, as indicated, for 40 hours and the cultures analyzed for DUX4, DUX4 target (MBD3L2, ZSCAN4 and LEUTX) or differentiation marker (MYOG and MYH2) RNA levels by qRT-PCR. Data is expressed as relative expression with the expression in absence of inhibitor set to one.

FIGS. 4A-4D show the modulation of DUX4 and DUX4 target gene expression by PH-797804 in a mouse xenograft model of FSHD. Human MB200 FSHD2 xenografts were created by co-injecting MB200 FSHD2 myoblasts with barium chloride into the tibialis anterior (TA) muscles of immunodeficient mice. PH-797804 was administered by subcutaneous injection at 10 mpk BID starting immediately after FSHD myoblast implantation. Seven days after implantation, RNA was isolated from TA muscles and analyzed for human-specific gene expression using qRT-PCR. FIG. 4A. hZSCAN gene expression. FIG. 4B. hMBD3L2 gene expression. FIG. 4C. hLEUTX gene expression. FIG. 4D. hDUX4 gene expression. Relative mRNA levels for each gene were normalized to that in the Vehicle control group, which was set to 1. Error bars indicate the standard error of the mean of eight biological replicates. P-values were calculated using an unpaired, two-tailed, two-sample t-test. *, p<0.05. **, p<0.01.

FIGS. 5A-B show that siRNAs targeting p38alpha or p38beta, alone or in combination, suppress DUX4 and DUX4 target gene expression. siRNAs were transfected into FSHD1 (FIG. 5A) and FSHD2 (FIG. 5B) myoblasts one day prior to differentiation. After differentiation for 40 hours, RNA was harvested and relative RNA levels were determined for p38alpha, p38beta, DUX4 and DUX4 targets (MBD3L2, ZSCAN4) by qRT-PCR. Data is expressed as relative expression with the expression of control siRNA (si-CTRL) set to one.

FIGS. 6A-6D show the modulation of DUX4 and DUX4 target gene expression by losmapimod in a mouse xenograft model of FSHD. Human MB200 FSHD2 xenografts were created by co-injecting MB200 FSHD2 myoblasts with barium chloride into the tibialis anterior (TA) muscles of immunodeficient mice. Losmapimod was administered by oral gavage at 2, 6 and 18 mpk BID starting immediately after FSHD myoblast implantation. Four days after implantation, RNA was isolated from TA muscles and analyzed for human-specific gene expression using qRT-PCR. FIG. 6A. hZSCAN gene expression. FIG. 6B. hMBD3L2 gene expression. FIG. 6C. hLEUTX gene expression. FIG. 6D. hDUX4 gene expression. Relative mRNA levels for each gene were normalized to that in the Vehicle control group, which was set to 1. Error bars indicate the standard error of the mean of 5-6 biological replicates. P-values were calculated using one-way analysis of variance followed by Dunnett's post test. *, p<0.05. ** p<0.01.

FIGS. 7A-7B show human cell content (survival) and human muscle differentiation marker expression and after long term (14 day) treatment with losmapimod in a mouse xenograft model of FSHD. Human MB200 FSHD2 xenografts were created by co-injecting MB200 FSHD2 myoblasts with barium chloride into the tibialis anterior (TA) muscles of immunodeficient mice. Losmapimod was administered by oral gavage at 6 mpk BID starting immediately after FSHD myoblast implantation. Fourteen days after implantation, RNA and DNA were isolated from TA muscles and analyzed for human DNA copy number (DNA) by qPCR and human-specific gene expression (RNA) using qRT-PCR. FIG. 7A. Human cell content. Relative human DNA content (two copy hTERT gene) was normalized first to total mouse DNA content (two copy mTfrc gene) and then the vehicle control was set to one. FIG. 7B. hMYH2 gene expression. Relative mRNA levels for hMYH2 were first normalized to the internal control hRPL30 gene and then to the Vehicle control group, which was set to 1. Error bars indicate the standard error of the mean of eight to twelve biological replicates. P-values were calculated using an unpaired, two-tailed, two-sample t-test.

### DESCRIPTION OF ILLUSTRATIVE EMBODIMENTS

Provided herein are inhibitors of p38 that show modulation of DUX4 expression and may be used in the treatment of facioscapulohumeral muscular dystrophy (FSHD). While Wnt/β-catenin pathway activators and estrogen have been shown to effect DUX4 expression, both of these molecules have potential to lead to off-target activities in other pathways. Inhibitors of p38 may be used to modulate the expression of DUX4 without triggering these other pathways. These and other embodiments will be described in more detail herein.

### I. FACIOSCAPULOHUMERAL MUSCULAR DYSTROPHY (FSHD)

Facioscapulohumeral dystrophy (FSHD or FSH) is a progressive muscle disease and a prevalent form of muscular dystrophy. The disease typically presents in young adults as facial and upper extremity weakness, and progresses to involve nearly all skeletal muscle groups [Tawil, et al., 2014]. FSHD is caused by the mis-expression of the double homeobox 4 (DUX4) transcription factor in skeletal muscle. DUX4 is encoded by a retrogene located in each unit of the D4Z4 macrosatellite repeat array in the subtelomeric region of chromosomes 4q and 10q. FSHD results from a contraction at 4q35 resulting in too few D4Z4 repeats for efficient repeat-mediated epigenetic repression (FSHD type 1, FSHD1) or from the presence of mutations in trans-acting chromatin factors necessary for epigenetic repression of the D4Z4 array (FSHD type 2, FSHD2) [Lemmers, et al., 2012, Lemmers, et al., 2010, van den Boogaard, et al., 2016]. Inefficient D4Z4 repression, when combined with a permissive chromosome 4qA haplotype that provides a polyadenylation site for the DUX4 messenger RNA (mRNA), results in the ectopic expression of DUX4 protein in muscle cells [Lemmers, et al., 2010, Snider, et al., 2010, Tawil, et al., 2014]. DUX4 is normally expressed in the pre-implantation embryo and in germline tissues, where it activates early developmental and stem cell genes [De Iaco, et al., 2017, Hendrickson, et al., 2017, Tawil, et al., 2014, Whiddon, et al., 2017]. In most somatic tissues, including skeletal muscle, the D4Z4 arrays and DUX4 are epigenetically silenced through multiple mechanisms that suppress repetitive elements in the genome [Das and Chadwick, 2016, Daxinger, et al., 2015, Snider, et al., 2010, van Overveld, et al., 2003, Zeng, et al., 2009]. DUX4 mis-expression in skeletal muscle induces early embryo, stem cell and germline genes; activates repetitive elements; suppresses innate immune response and nonsense-mediated RNA decay pathways; inhibits myogenesis; and causes cell death through mechanisms that include the accumulation of aberrant and double-stranded RNAs [Bosnakovski, et al., 2008, Feng, et al., 2015, Geng, et al., 2012, Kowaljow, et al., 2007, Rickard, et al., 2015, Shadle, et al., 2017, Snider, et al., 2009, Wallace, et al., 2011, Winokur, et al., 2003, Young, et al., 2013].

FSHD gets its name from the major muscle groups which are involved, namely the face, shoulders and upper arms. In general, muscle weakness starts in the face and moves down the body to the shoulders and upper arms followed by the weakening of the lower arms, abdominal muscles, and the hips and leg muscles. Additionally, patients with FSHD may also experience abnormal heart rhythm, hearing loss, and foot drop. FSHD, as a genetic disease, is typically and definitively diagnosed with a genetic test. Other tests such as creatine kinase levels, electromyogram, nerve conduction velocity, and muscle biopsy may be used to test for FSHD in some clinical situations but none of these tests are as accurate or specific for FSHD as genetic testing. Currently, there are no treatments for FSHD specifically, but palliative treatments which target pain and loss of function associated with muscle wasting. These treatments may include therapy such as physical therapy or occupational therapy, and nonsteroidal anti-inflammatory or opioid pain medications. Alternatively, surgical interventions such as scapular fusion or bracing may be used to improve the patient's quality of life. Therapeutic agents in current clinical trials target only general increases in muscle mass (e.g. ACE-083) or a potential immune component (e.g. Resolaris/ATYR1940) without addressing the underlying cause of muscle degeneration, DUX4 expression.

### II. P38 INHIBITORS

As used herein, the abbreviation “p38” refers to any of the p38 mitogen-activated protein (MAP) kinases. p38 is a mammalian protein kinase involved cell proliferation, cell death and response to extracellular stimuli. Activation of p38 has been observed in cells stimulated by stresses, such as treatment of lipopolysaccharides (LPS), UV, anisomycin, or osmotic shock, and by cytokines, such as IL-1 and TNF. Inhibition of p38 kinase leads to a blockade on the production of both IL-1 and TNF. IL-1 and TNF stimulate the production of other proinflammatory cytokines such as IL-6 and IL-8 and have been implicated in acute and chronic inflammatory diseases and in postmenopausal osteoporosis [Kimble, et al., 1995]. Several p38 MAP kinases have been identified, including p38-α (also known as MAPK14), p38-β(also known as MAPK11), p38-γ (also known as MAPK12/ERK6) and p38-δ (also known as MAPK13/SAPK4). The nucleic acid sequences of the genes encoding p38, including, but not limited to, the nucleic acid sequences of the open reading frames of the genes, are known in the art. The amino acid sequences of p38 polypeptides and proteins, including, but not limited to, the amino acid sequences of the human p38 polypeptides and proteins, are known in the art. The accession number of the nucleic acid sequence of Mus musculus p38-α (MAPK14) is NM_011951.3 and the accession number of the nucleic acid sequence of human p38-α (MAPK14) is NM_001315.2. The accession number of the amino acid sequence of Mus musculus p38-α (MAPK14) is NP_036081.1 and the accession number of the amino acid sequence of human p38-α (MAPK14) is NP_001306.1. For additional information on p38, see [Cuadrado and Nebreda, 2010, Kostenko, et al., 2011, Marber, et al., 2011], all of which are incorporated by reference herein.

In various embodiments described herein, the term “p38 inhibitor” may be used to refer to a molecule that reduces a biological activity of p38. A p38 inhibitor may be any member of a class of compounds (e.g., small molecule or antibody) that reduces a biological activity (e.g., kinase activity) of a p38 protein (e.g., p38α, p38β, p38γ, or p38δ). A p38 inhibitor may also be any member of a class of compounds that decreases the expression of a nucleic acid encoding a p38 (e.g., an inhibitory nucleic acid such as RNAi). Preferably, a p38 inhibitor is a compound that exhibits p38-inhibitory activity in a suitable p38 biological assay, e.g., an assay measuring the conversion of ATP to ADP or phosphorylation of a target protein/peptide. See, e.g., the ADP-Glo™ p38α, p38β, p38γ, or p38δ Kinase Assays available from Promega Corp. The skilled artisan is able to determine whether a compound would qualify as a p38 inhibitor in such an assay. Preferably, a p38 inhibitor is a compound that exhibits more than 50% inhibition of the activity of one or more p38 proteins in a p38 assay at 500 nM, more preferably one hat exhibits more than 50% inhibition of the activity of one or more p38 proteins in a p38 assay at 250 nM, still more preferably one that exhibits more than 50% inhibition of the activity of one or more p38 proteins in a p38 assay at 100 nM, and even more preferably one that exhibits more than 50% inhibition of the activity of one or more p38 proteins in a p38 assay at 50 nM or less.

Both pan p38 inhibitors and specific inhibitors of one isoform of p38 are contemplated herein and may be used to treat FSHD.

In some embodiments, the p38 inhibitor may be a selective inhibitor of p38α. The phrases “selective p38α inhibitor” or “selective inhibitor of p38α” may be used to refer to a compound that has an IC₅₀ value for p38α that is at least two-fold lower, or at least three-fold lower, than its IC₅₀ values for p38β, p38γ, and p388.

In some embodiments, the p38 inhibitor may be a selective inhibitor of p38β. The phrases “selective p38β inhibitor” or “selective inhibitor of p38β” may be used to refer to a compound that has an IC₅₀ value for p38β that is at least two-fold lower than its IC₅₀ values for p38α, p38γ, and p388.

A non-limiting selections selection of different inhibitors of p38 or related pathways are shown in Table 1 below.

TABLE 1
Non-limiting List of Inhibitors in differentiating
cultures of FSHD1 and FSHD2 muscle cells

| Inhibitor | Mechanism/Selectivity |
|---|---|
| Acumapimod | p38α/β |
| ARRY-371797 | p38α |
| BMS-582949 | p38α, 5X selective over p38β |
| Dilmapimod | p38α/β |
| Dorimapimod | p38α; 20X selective over B-Raf |
| eFT-508 | MNK 1/2 |
| Losmapimod | p38α/β |
| LY2228820 | p38α/β |
| LY3007113 | p38 |
| Pamapimod | p38α, 34X selective over p38β |
| PF-3644022 | MAPKAPK2 (MK2) |
| PH-797804 | p38α, 4X selective over p38β |
| SB202190 | p38α/β |
| SB203580 | p38α/β |
| TAK-715 | p38α, 28X selective over p38β |
| Talmapimod | p38α, 10X selective over p38β |
| VX-702 | p38α, 14X selective over p38β |
| VX-745 | p38α, 22X selective over p38β |

Additional p38 inhibitors which may be used herein include those described in [Fisk, et al., 2014, Gangwal, et al., 2013, Karcher and Laufer, 2009, Kumar, et al., 2003, Lee, et al., 2000, Norman, 2015, Yong, et al., 2009], WO 2003/068747, WO 2003/093248, US 2005/0020540, US 2006/0122221, US 2005/0176964, US 2004/0267012, WO 2005/012241, WO 2004/010995, WO 2005/073189, US 2004/0102636, US 2004/0132729, US 2005/0020590, WO 1999/032463, US 2003/0232831, WO 2000/012497, US 2002/0118671, WO 2000/012074, WO 2010/067131, WO 1999/000357, US 2004/0254236, U.S. Pat. No. 5,945,418, US 2009/0143422, US 2009/0136596, US 2007/0185175, US 2009/0118272, US 2007/0213300, US 2008/0171741, US 2010/0215652, US 2011/0190292, WO 2009/155388, US 2011/0077243, WO 2012/031057, WO 2009/155388, and U.S. Pat. No. 8,252,818, the entirety of which are herein by incorporated by reference.

### III. THERAPEUTIC METHODS

In another aspect, this disclosure provides methods of modulating DUX4 expression using p38 inhibitors such as one or more of the compounds disclosed herein, as well as pharmaceutical compositions thereof. Such pharmaceutical compositions further comprise one or more non-toxic, pharmaceutically acceptable carriers and/or diluents and/or adjuvants (collectively referred to herein as “carrier” materials) and if desired other active ingredients. In some embodiments, the compound is administered as part of a pharmaceutical composition further comprising a pharmaceutically acceptable carrier. In some embodiments, the compounds and/or pharmaceutical compositions thereof may be administered orally, parenterally, or by inhalation spray, or topically in unit dosage formulations containing conventional pharmaceutically acceptable carriers, adjuvants and vehicles. The term parenteral as used herein includes, for example, subcutaneous, intravenous, intramuscular, intrasternal, infusion techniques or intraperitoneally. In some embodiments, the compounds of the present disclosure are administered by any suitable route in the form of a pharmaceutical composition adapted to such a route, and in a dose effective for the treatment intended. Therapeutically effective doses of the compounds required to prevent or arrest the progress of or to treat a medical condition are readily ascertained by one of ordinary skill in the art using preclinical and clinical approaches familiar to the medicinal arts.

Based upon standard laboratory experimental techniques and procedures well known and appreciated by those skilled in the art, as well as comparisons with compounds of known usefulness, the compounds described above can be used in the treatment of patients suffering from the above pathological conditions. One skilled in the art will recognize that selection of the most appropriate compound of the disclosure is within the ability of one with ordinary skill in the art and will depend on a variety of factors including assessment of results obtained in standard assay and animal models.

### IV. PHARMACEUTICAL FORMULATIONS AND ROUTES OF ADMINISTRATION

For administration to a primate, especially a human, in need of such treatment, the compounds in a therapeutically effective amount are ordinarily combined with one or more excipients appropriate to the indicated route of administration. The compounds of the present disclosure are contemplated to be formulated in a manner amenable to treatment of a veterinary patient as well as a human patient. In some embodiments, the veterinary patient may be a non-human primate. The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol, and tableted or encapsulated for convenient administration. Alternatively, the compounds may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art and may be adapted to the type of primate being treated.

The pharmaceutical compositions useful in the present disclosure may be subjected to conventional pharmaceutical operations such as sterilization and/or may contain conventional pharmaceutical carriers and excipients such as preservatives, stabilizers, wetting agents, emulsifiers, buffers, etc.

The compounds of the present disclosure may be administered by a variety of methods, e.g., orally or by injection (e.g. subcutaneous, intravenous, intraperitoneal, etc.). Depending on the route of administration, the active compounds may be coated in a material to protect the compound from the action of acids and other natural conditions which may inactivate the compound. They may also be administered by continuous perfusion/infusion of a disease or wound site.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The therapeutic compound may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions may be suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it may be useful to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the methods of preparation include vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general a human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

An effective amount typically will vary from about 0.001 mg/kg to about 1000 mg/kg, from about 0.01 mg/kg to about 750 mg/kg, from about 100 mg/kg to about 500 mg/kg, from about 1.0 mg/kg to about 250 mg/kg, from about 10.0 mg/kg to about 150 mg/kg in one or more dose administrations daily, for one or several days (depending of course of the mode of administration and the factors discussed above). Other suitable dose ranges include 1 mg to 10000 mg per day, 100 mg to 10000 mg per day, 500 mg to 10000 mg per day, and 500 mg to 1000 mg per day. In some particular embodiments, the amount is less than 10,000 mg per day with a range of 750 mg to 9000 mg per day.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day. For example, regarding treatment of diabetic patients, the unit dosage may be an amount that reduces blood glucose by at least 40% as compared to an untreated subject. In another embodiment, the unit dosage is an amount that reduces blood glucose to a level that is +10% of the blood glucose level of a non-diabetic subject.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In an exemplary embodiment, the pharmaceutical composition comprises losmapimod. In some embodiments, the pharmaceutical composition comprises losmapimod at a dose of 1 mg to 100 mg. In more specific embodiments, the pharmaceutical composition comprises losmapimod at a dose of 3 mg to 25 mg. In further specific embodiments, the pharmaceutical composition comprises losmapimod at a dose of 5 mg to 10 mg. In some embodiments, the pharmaceutical composition comprises losmapimod at a dose of 2.5 mg, 5 mg, 7.5 mg, 10 mg, 12.5 mg, 15 mg, 17.5 mg, or 20 mg. In an exemplary embodiment, the pharmaceutical composition comprises losmapimod at a dose of 7.5 mg. In certain embodiments, an FSHD patient in need of treatment is administered a pharmaceutical composition comprising losmapimod once daily. In certain other embodiments, an FSHD patient in need of treatment is administered a pharmaceutical composition comprising losmapimod twice daily. In other embodiments, an FSHD patient in need of treatment is administered a pharmaceutical composition comprising losmapimod three, four, or more times daily. In some embodiments, an FSHD patient in need of treatment is administered a pharmaceutical composition comprising 2.5 mg, 5 mg, 7.5 mg, 10 mg, 12.5 mg, or 15 mg losmapimod twice daily. In an exemplary embodiment, an FSHD patient in need of treatment is administered a pharmaceutical composition comprising 7.5 mg losmapimod twice daily. In another exemplary embodiment, an FSHD patient in need of treatment is administered a pharmaceutical composition comprising 15 mg losmapimod twice daily.

In certain embodiments, a pharmaceutical composition of the present disclosure may comprise, for example, at least about 0.1% of a compound of the present disclosure. In other embodiments, the compound of the present disclosure may comprise between about 1% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the subject has eaten or will eat.

### V. DEFINITIONS

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

An “active ingredient” (AI) (also referred to as an active compound, active substance, active agent, pharmaceutical agent, agent, biologically active molecule, or a therapeutic compound) is the ingredient in a pharmaceutical drug or a pesticide that is biologically active. The similar terms active pharmaceutical ingredient (API) and bulk active are also used in medicine, and the term active substance may be used for pesticide formulations.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease. For the purposes of this invention, an effective amount of a p38 inhibitor is an amount sufficient to slow the progression of one or more symptoms of FSHD, including, but not limited to, facial muscle weakness, shoulder weakness, hearing loss, abnormal heart rhythm, weakening of muscles in the upper body, loss of strength in the abdominal muscles, or foot drop.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular active ingredient or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug) is a compound or composition used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active ingredient according to the present disclosure. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any of the reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Methods and Materials

Cells and Cell Culture.

Primary human myoblast cell lines were obtained from the Fields Center at the University of Rochester (world-wide-web at urmc.rochester.edu/fields-center.aspx) and immortalized by retroviral transduction of cyclin-dependent kinase 4 (CDK4) and human telomerase reverse transcriptase (hTERT) [Stadler, et al., 2011]. Immortalized myoblasts were grown in Ham's F-10 Nutrient Mix (Gibco, Waltham, Mass., USA) supplemented with 20% HyClone Fetal Bovine Serum (GE Healthcare Life Sciences, Pittsburgh, Pa., USA), 100 U/100 μg penicillin/streptomycin (Gibco), 10 ng/ml recombinant human fibroblast growth factor (Promega Corporation, Madison, Wis., USA) and 1 μM dexamethasone (Sigma-Aldrich). Differentiation of myoblasts into myotubes was achieved by switching the fully confluent myoblast monolayer into Dulbecco's Modified Eagle Medium (DMEM) (Gibco) containing 1% horse serum (Gibco), 100 U/100 μg penicillin/streptomycin, g/ml insulin (Sigma-Aldrich) and 10 μg/ml transferrin (Sigma-Aldrich) (HS/IT media) or DMEM:Nutrient Mixture F-12 (1:1, Gibco) supplemented with 2% KnockOut Serum Replacement (Gibco), 100 U/100 μg penicillin/streptomycin, 10 μg/ml insulin and 10 μg/ml transferrin (KSR media) for 2-6 days.

qPCR Analysis of MBDL32, MYOG and MYH2 Expression.

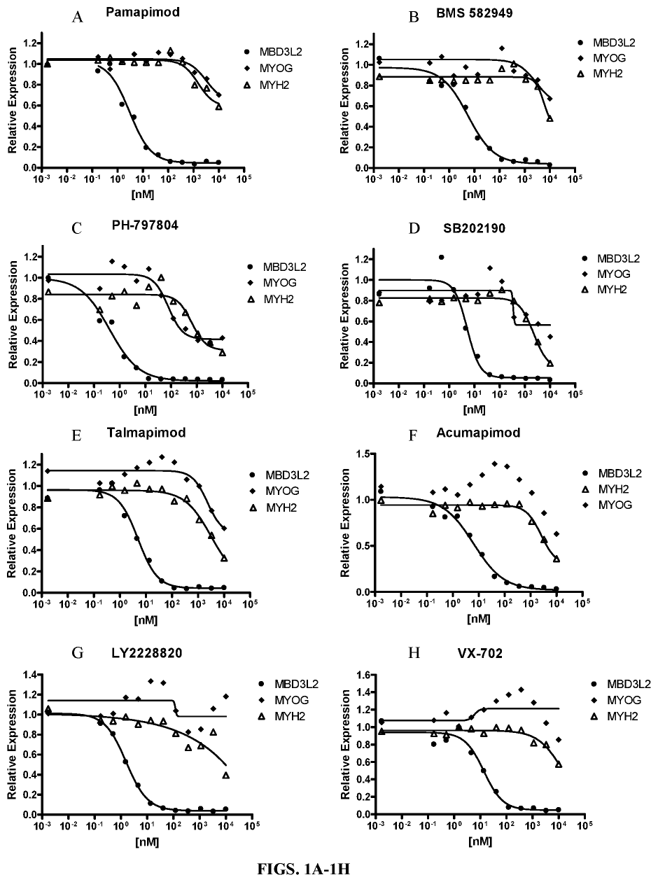

Differentiating MB200 FSHD2 muscle cells were exposed to p38 inhibitors (Table 2) at a variety of concentrations for 40 hours to determine the effects of the drugs on DUX4 expression. Gene expression of MBDL32, a target gene of DUX4, was measured by qPCR (FIG. 1). For screening compounds in FSHD1 and FSHD2 muscle cells, cell lysates were prepared using Cells-to-Ct Bulk Lysis Reagents (Invitrogen, Waltham, Mass., USA). Quantitative polymerase chain reaction (PCR) was carried out on a QuantStudio 5 (Applied Biosystems, Waltham, Mass., USA) using TaqMan Gene Expression Assays (Applied Biosystems) and TaqMan Fast Virus 1-Step Master Mix (Invitrogen). The relative expression levels of DUX4 target gene methyl-CpG binding domain protein 3 like 2 (MBD3L2), differentiation marker myosin heavy chain (MYH2) or differentiation marker myogenin (MYOG) was normalized to that of the reference gene ribosomal protein L30 (RPL30), which was included in multiplex (two gene) PCR reactions. TaqMan Gene Expression Assay ID numbers: MBD3L2, Hs00544743_m1; MYH2, Hs00430042_m1; MYOG, Hs01072232_m1; RPL30, Hs00265497_m1. Data is expressed as relative expression with the expression in absence of inhibitors set to one.

TABLE 2
Potency of Inhibitors in differentiating
cultures of FSHD1 and FSHD2 muscle cells.

| | | FSHD1 | FSHD2 |
|---|---|---|---|
| | | EC₅₀ | EC₅₀ |
| Inhibitor | Mechanism/Selectivity | (nM) | (nM) |
| Acumapimod | p38α/β | 13 | 6.3 |
| BMS-582949 | p38α, 5X selective over p38β | 17 | 4.2 |
| eFT-508 | MNK 1/2 | NT | >10,000 |
| LY2228820 | p38α/β | 4.0 | 1.6 |
| Pamapimod | p38α, 34X selective over p38β | 5.5 | 2.2 |
| Losmapimod | | 14 | 2.4 |
| PF-3644022 | MAPKAPK2 (MK2) | NT | >10,000 |
| PH-797804 | p38α, 4X selective over p38β | 0.15 | 0.41 |
| SB202190 | p38α/β | 0.57 | 5.2 |
| SB203580 | p38α/β | NT | <5,000 |
| TAK-715 | p38α, 28X selective over p38β | 4.4 | 12 |
| Talmapimod | p38α, 10X selective over p38β | 1.6 | 5.0 |
| VX-702 | p38α, 14X selective over p38β | 4.1 | 14 |
| VX-745 | p38α, 22X selective over p38β | 12 | 13 |

*NT, not tested

qPCR Analysis of DUX4 and LEUTX Expression.

FSHD2 MB200 myoblasts were induced to differentiate for 48 hours. The cultures were then treated in triplicate with the p38 inhibitor SB203580 (10 μM) or DMSO as a control for the last 16, 20 or 24 hours of the 48 hour differentiation period. Expression of DUX4, LEUTX, Desmin, and RPL27 were measured by qPCR as follows: total RNA was extracted from whole cells using the RNeasy Mini Kit (Qiagen, Hilden, Germany) according to the manufacturer's instructions. Isolated RNA was treated with DNase I (Thermo Fisher Scientific), heat inactivated and reverse transcribed into cDNA using Superscript III (Thermo Fisher Scientific) and oligo(dT) primers (Invitrogen) following the manufacturer's protocol. Quantitative PCR was carried out on a QuantStudio 7 Flex (Applied Biosystems) using primers specific for each mRNA and iTaq SYBR Green Supermix (Bio-Rad Laboratories, Inc., Hercules, Calif., USA). The relative expression levels of target genes were normalized to that of the reference genes ribosomal protein L27 (RPL27) by using the delta-delta-Ct method [Livak and Schmittgen, 2001] after confirming equivalent amplification efficiencies of reference and target molecules. Primers are as follows:

(SEQ ID NO: 1)
DUX4 F: GGCCCGGTGAGAGACTCCACAC;
(SEQ ID NO: 2)
DUX4 R: CCAGGAGATGTAACTCTAATCCAGGTTTGC;
(SEQ ID NO: 3)
RPL27 F: GCAAGAAGAAGATCGCCAAG;
(SEQ ID NO: 4)
RPL27 R: TCCAAGGGGATATCCACAGA;
(SEQ ID NO: 5)
LEUTX F: TGGCTACAATGGGGAAACTG;
(SEQ ID NO: 6)
LEUTX R: CTGCTGCCTCTTCCATTTG;
(SEQ ID NO: 7)
DESMIN F: GATCAATCTCCCCATCCAGA;
(SEQ ID NO: 8)
DESMIN R: TGGCAGAGGGTCTCTGTCTT.

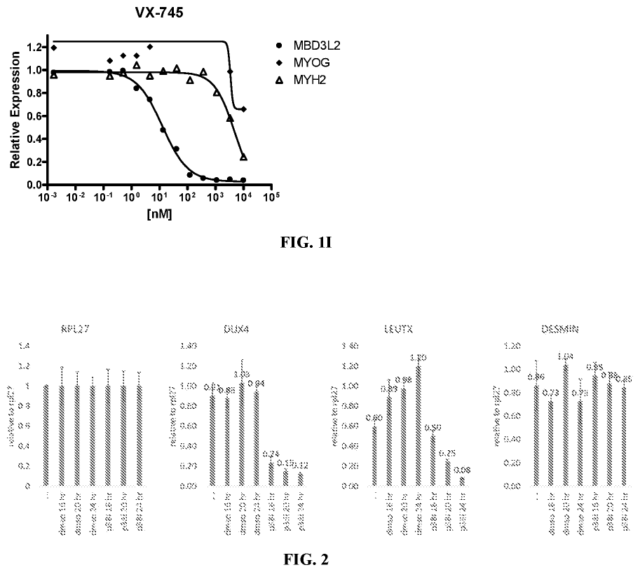

(FIG. 2). RPL27 was used as a control gene, unaffected by treatment, while Desmin is a differentiation marker.

Measurement of DUX4 and DUX4 Target Gene Expression in FSHD1 and FSHD2 Myotubes.

FSHD1 and FSHD2 myoblasts were induced to differentiate into myotubes for 40 hours in the presence of PH-797804. Total RNA was extracted from whole cells using the RNeasy Mini Kit (Qiagen, Hilden, Germany) according to the manufacturer's instructions. Isolated RNA was treated with DNase I (Thermo Fisher Scientific), heat inactivated and reverse transcribed into cDNA using Superscript III (Thermo Fisher Scientific) and oligo(dT) primers (Invitrogen) following the manufacturer's protocol. qPCR was performed on cDNA to measure expression of DUX4, MBDL32, ZSCAN4, LEUTX, MYOG, and MYH2 using TaqMan Gene Expression Assay ID numbers: MBD3L2, Hs00544743_m1; MYH2, Hs00430042 ml; MYOG, Hs01072232 ml; RPL30, Hs00265497_m1; LEUTX, Hs01028718_m1; ZSCAN4, Hs00537549_m1; or

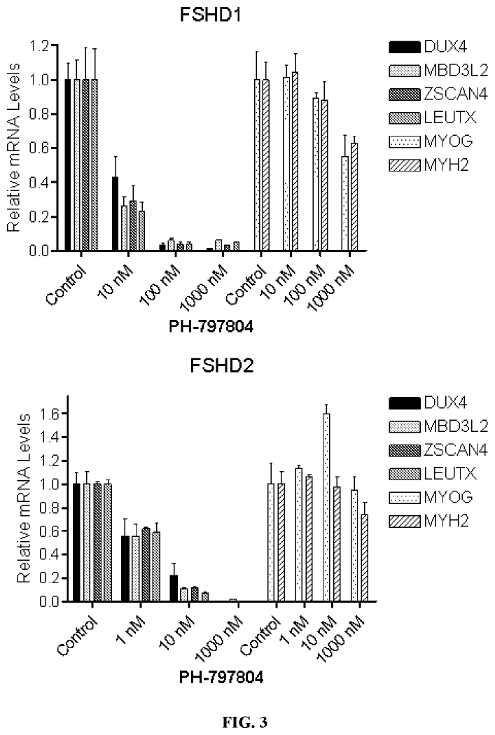

DUX4 with primers
(SEQ ID NO: 9)
GCCGGCCCAGGTACCA
and
(SEQ ID NO: 10)
CAGCGAGCTCCCTTGCA
with probe
(SEQ ID NO: 11)
6FAMCAGTGCGCACCCCGMGBNFQ (FIG. 3).

Analysis of DUX4 and Other Human Gene Expression in Xenograft Mice.

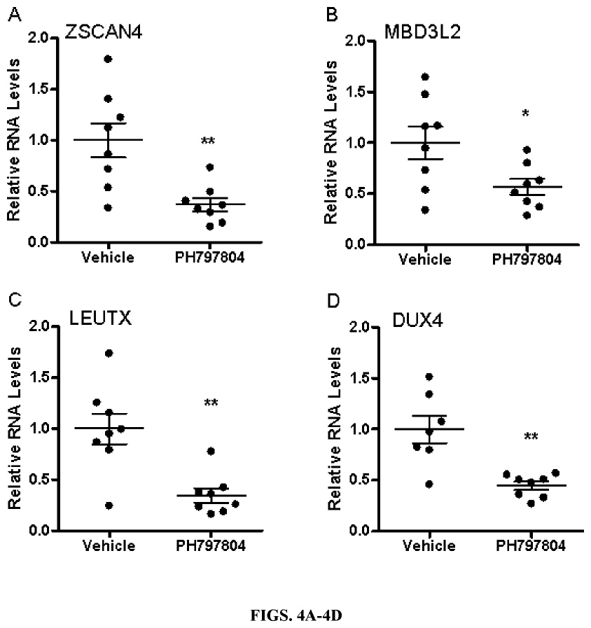

Human MB200 FSHD2 myoblasts co-injected with barium chloride into the tibialis anterior muscles of immunodeficient mice [Hardy, et al., 2016]. The grafted myoblasts became part of mature muscle myofibers in the mouse tibialis anterior muscle while retaining the genomic organization of human FSHD cells and the FSHD-specific regulation of DUX4. MB200 FSHD2 xenografted mice were administered PH-797804 by subcutaneous injection twice daily or losmapimod by oral gavage twice daily, beginning immediately after FSHD myoblast implantation. Four, seven or fourteen days post implantation, RNA was isolated from TA muscles. For 14 day samples, DNA was also purified. Purified RNA was analyzed for human specific gene expression by qRT-PCR using primers directed to hMYH2, hZSCAN, hMBD3L2, hLEUTX, and hDUX4 as described above (FIGS. 4A-D). Relative mRNA levels were normalized to that of the vehicle control group. For DNA analysis, human 2 copy gene hTERT levels and mouse two copy gene mTfrc DNA levels were determined by qPCR. Relative human cell numbers were determined by normalizing hTERT levels to total mTfrc levels. TaqMan Copy Number Reference Assay catalog numbers (ThermoFisher Scientific): hTERT, 4403316; mTfrc, 4458366.

### Example 2: Treatment with p38 Inhibitors Decreases DUX4 Expression

Analysis of MBD3L2 Expression in Cultured FSHD2 Cells.

DUX4 mRNA is present in very low levels, even in differentiating FSHD2 myoblasts, so DUX4 target gene MBD3L2 was analyzed for expression during differentiation of myoblasts into myotubes. FSHD2 patient derived myotubes were analyzed for MBD3L2 RNA levels to determine whether p38 inhibitors affect MBD3L2 expression, indicating a reduction in DUX4 expression. Additionally, expression of differentiation markers MYOG and MYH2 was examined to determine whether p38 inhibitors affect differentiation. As seen in FIGS. 1A-I, treatment with a variety of p38 inhibitors causes significant reductions in MBD3L2 expression at concentrations that do not affect the expression of differentiation markers. Potency of inhibitors was also determined (Table 2).

p38 Inhibitor SB203580 Reduces DUX4 Expression in FSHD Myotubes.

Cultured FSHD myoblasts were induced to differentiate for 48 hours and treated with p38 inhibitor SB203580 or DMSO for the time indicated (FIG. 2). Expression of RPL27, DUX4, LEUTX, and DESMIN was measured to determine the effect of a p38 inhibitor directly on expression of DUX4. SB203580 has a direct effect, reducing DUX4 expression. Reduced expression of DUX4 was further reflected in reduced expression of LEUTX, while the differentiation marker Desmin was not affected.

PH-797804 Treatment Reduces DUX4 Expression in FSHD Myotubes.

FSHD1 and FSHD2 myoblasts were induced to differentiate into myotubes in the presence of multiple concentrations of the Phase II clinical p38 inhibitor PH-797804. Expression of DUX4 was significantly decreased in the presence of PH-797804 (FIG. 3). DUX4 target genes MBD3L2, ZSCAN4, and LEUTX were similarly affected, all with significant decreases in expression. Differentiation markers MYOG and MYH2 were not affected by treatment with up to 100 nM PH-797804.

PH-797804 Reduces DUX4 Expression in FSHD Myoblast Xenografts.

To further demonstrate the potential clinical utility of p38 inhibition for FSHD, PH-797804 was tested in a mouse pharmacology model of human FSHD gene regulation wherein FSHD myoblasts were engrafted into the mouse tibialis anterior muscles. The grafted human myoblasts become part of mature muscle myofibers in the mouse TA muscle yet retain the genomic organization of human FSHD cells and maintain the unique FSHD-specific regulation of DUX4. Treatment of mice containing human MB200 FSHD2 xenografts with PH-797804 results in significant decreases in the expression of DUX4 and DUX4 target genes (FIGS. 4A-D), supporting the concept of systemic administration of a p38 inhibitor to target DUX4 expression in muscle tissue for the treatment of FSHD.

p38alpha and p38beta Each Play a Distinct Role to Promote DUX4 Expression in FSHD.

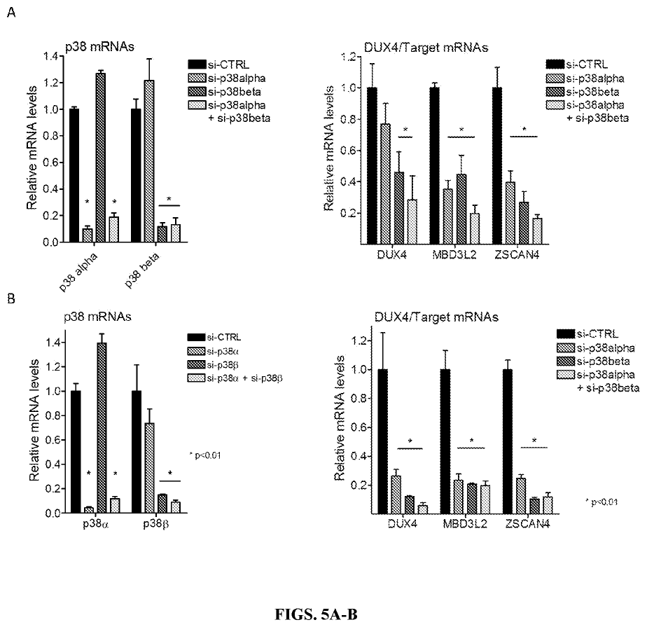

To genetically identify p38 isoforms involved in promoting DUX4 expression, siRNAs targeting p38alpha and p38beta were transfected into FSHD1 (54-2) and FSHD2 (MB200) myoblasts before differentiation into myotubes. In FSHD1 (FIG. 5A) and FSHD2 (FIG. 5B) myotubes, siRNAs targeting p38alpha, p38beta or p38alpha in combination with p38beta resulted in reduced expression of DUX4 and its target genes MBD3L2 and ZSCAN4. These results demonstrate that p38alpha and p38beta play distinct essential roles in promoting DUX4 expression and that each is a potential drug target for selective inhibitor development for FSHD.

Losmapimod Reduces DUX4 Expression in FSHD Myoblast Xenografts.

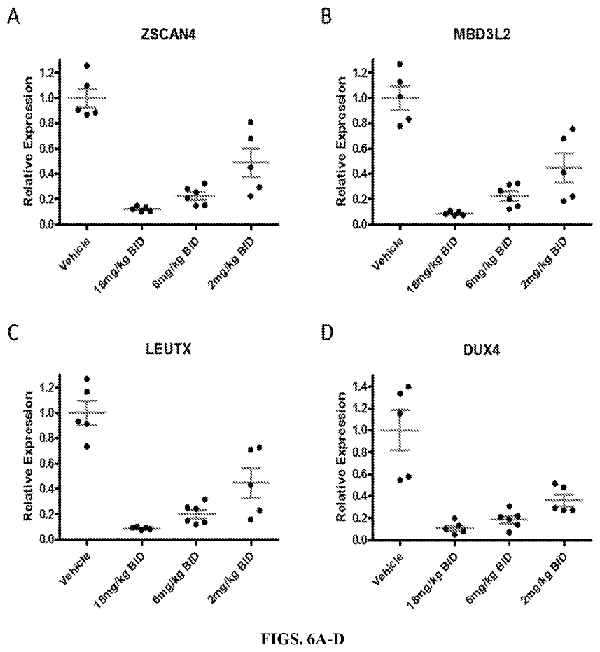

To further demonstrate the potential clinical utility of p38 inhibition for FSHD, Losmapimod was tested in the mouse xenograft pharmacology model of human FSHD gene regulation (as in FIG. 4). Treatment of mice containing human MB200 FSHD2 xenografts with Losmapimod resulted in significant decreases in the expression of DUX4 and DUX4 target genes in a dose-dependent manner (FIGS. 6A-D), supporting the concept of systemic administration of a p38 inhibitor, and specifically losmapimod, to target DUX4 expression in muscle tissue for the treatment of FSHD.

Losmapimod Treatment Promotes FSHD Cell Survival and Engraftment in Xenograft Mice.

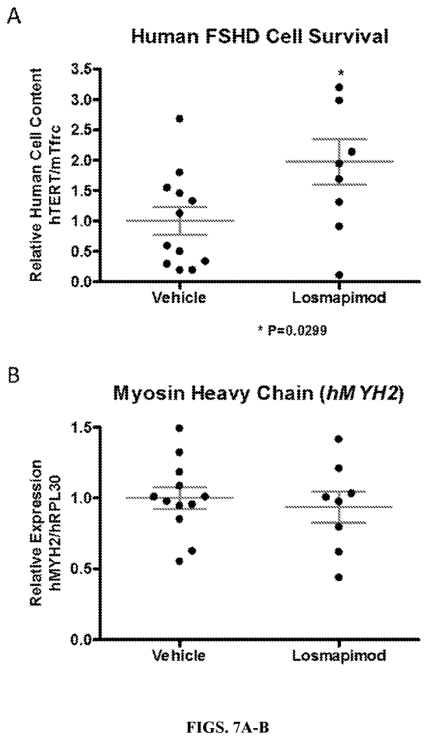

To further demonstrate the potential clinical utility of p38 inhibition for FSHD, Losmapimod was tested in the mouse FSHD xenograft model over an extended fourteen day time period. After fourteen days, xenograft have matured and result in fully formed muscle myofibers [Hardy, et al., 2016]. Treatment of mice containing human MB200 FSHD2 xenografts with Losmapimod for fourteen days starting immediately after xenotransplantation resulted in a significant increase in the survival of human FSHD cells based on DNA copy number (FIG. 7A). Losmapimod treatment did not affect the relative expression differentiation marker hMYH2 which is normalized to the human internal control gene hRPL30 (FIG. 7B) This results indicates that surprisingly, Losmapimod treatment enhanced the survival and engraftment of fully differentiated FSHD cells, further supporting the concept of systemic administration of a p38 inhibitor, and specifically Losmapimod, for the treatment of FSHD.

All of the compounds, compositions, and methods disclosed and claimed herein can be made and executed without undue experimentation in light of the present disclosure. While the disclosure may have focused on several embodiments or may have been described in terms of preferred embodiments, it will be apparent to those of skill in the art that variations and modifications may be applied to the compounds, compositions, and methods without departing from the spirit, scope, and concept of the disclosure. All variations and modifications apparent to those skilled in the art are deemed to be within the spirit, scope, and concept of the disclosure as defined by the appended claims.

### REFERENCES

The following references to the extent that they provide exemplary procedural or other details supplementary to those set forth herein, are specifically incorporated herein by reference.
US 2002/0118671
US 2003/0232831
US 2004/0254236
US 2004/0267012
US 2004/0102636
US 2004/0132729
US 2005/0020590
US 2005/0020540
US 2006/0122221
US 2007/0185175
US 2007/0213300
US 2008/0171741
US 2009/0143422
US 2009/0136596
US 2009/0118272
US 2011/0190292
US 2011/0077243
U.S. Pat. No. 5,945,418
WO 1999/032463
WO 1999/000357
WO 2000/012497
WO 2000/012074
WO 2003/068747
WO 2003/093248
WO 2004/010995
WO 2005/012241
WO 2005/073189
WO 2009/155388
WO 2009/155388
WO 2010/067131
WO 2012/031057
Bosnakovski, D., et al., The EMBO Journal 2008, 27 (20), 2766-79.
Cuadrado, A.; Nebreda, A. R., Biochem J 2010, 429 (3), 403-17.
Das, S.; Chadwick, B. P., PLoS One 2016, 11 (7), e0160022.
Daxinger, L., et al., Curr Opin Genet Dev 2015, 33, 56-61.
De Iaco, A., et al., Nat Genet 2017, 49 (6), 941-945.
Feng, Q., et al., Elife 2015, 4.
Fisk, M., et al., Am J Cardiovasc Drugs 2014, 14 (3), 155-65.
Gangwal, R. P., et al., Curr Top Med Chem 2013, 13 (9), 1015-35.
Geng, L. N., et al., Developmental Cell 2012, 22 (1), 38-51.
Hardy, D., et al., PloS One 2016, 11 (1), e0147198.
Hendrickson, P. G., et al., Nat Genet 2017, 49 (6), 925-934.
Karcher, S. C.; Laufer, S. A., Curr Top Med Chem 2009, 9 (7), 655-76.
Kimble, R. B., et al., Endocrinology 1995, 136 (7), 3054-61.
Kostenko, S., et al., World J Biol Chem 2011, 2 (5), 73-89.
Kowaljow, V., et al., Neuromuscular Disorders: NMD 2007, 17 (8), 611-23.
Kumar, S., et al., Nature reviews. Drug Discovery 2003, 2 (9), 717-26.
Lee, J. C., et al., Immunopharmacology 2000, 47 (2-3), 185-201.
Lemmers, R. J., et al., Nature Genetics 2012, 44 (12), 1370-4.
Lemmers, R. J., et al., Science 2010, 329 (5999), 1650-3.
Livak, K. J.; Schmittgen, T. D., Methods 2001, 25 (4), 402-8.
Marber, M. S., et al., Journal of Molecular and Cellular Cardiology 2011, 51 (4), 485-90.
Norman, P., Expert Opin Investig Drugs 2015, 24 (3), 383-92.
Rickard, A. M., et al., Human Molecular Genetics 2015, 24 (20), 5901-14.
Shadle, S. C., et al., PLoS Genet 2017, 13 (3), e1006658.
Snider, L., et al., Human Molecular Genetics 2009, 18 (13), 2414-30.
Snider, L., et al., PLoS Genetics 2010, 6 (10), e1001181.
Stadler, G., et al., Skelet Muscle 2011, 1 (1), 12.
Tawil, R., et al., Skeletal muscle 2014, 4, 12.
van den Boogaard, M. L., et al., American Journal of Human Genetics 2016, 98 (5), 1020-9.
van Overveld, P. G., et al., Nature Genetics 2003, 35 (4), 315-7.
Wallace, L. M., et al., Annals of Neurology 2011, 69 (3), 540-52.
Whiddon, J. L., et al., Nat Genet 2017, 49 (6), 935-940.
Winokur, S. T., et al., Hum Mol Genet 2003, 12 (22), 2895-907.
Yong, H. Y., et al., Expert Opin Investig Drugs 2009, 18 (12), 1893-905.
Young, J. M., et al., PLoS Genetics 2013, 9 (11), e1003947.
Zeng, W., et al., PLoS Genetics 2009, 5 (7), e1000559.

1. A method of treating a patient with facioscapulohumeral muscular dystrophy (FSHD) comprising administering to the patient a therapeutically effective amount of an inhibitor of p38.

2. The method of claim 1, wherein the inhibitor of p38 is an inhibitor of p38α and p38β.

3. The method of claim 1, wherein the inhibitor of p38 is a selective inhibitor of p38α.

4. The method of claim 1, wherein the inhibitor of p38 is a selective inhibitor of p38β.

5. The method according to claim 1, wherein the inhibitor of p38 modulates the expression of DUX4.

6. The method according to claim 1, wherein the inhibitor of p38 does not inhibit the MK2 pathway.

7. The method according to claim 1, wherein the inhibitor of p38 does not inhibit either p38δ or p38γ.

8. The method according to claim 1, wherein the inhibitor of p38 is selected from acumapimod, ARRY-371797, pexmetinib, AS1940477, BMS-582949, dilmapimod, dorimapimod, losmapimod, LY2228820, LY3007113, pamapimod, PH-797804, SB202190, SB203580, TAK-715, talmapimod, VX-702, and VX-745.

9. The method according to claim 1, wherein the FSHD has been diagnosed.

10. The method according to claim 1, wherein the FSHD has not been diagnosed.

11. The method according to claim 1, wherein the FSHD is adult-onset FSHD.

12. The method according to claim 1, wherein the FSHD is infantile-onset FSHD.

13. The method according to claim 1, wherein the FSHD is Type 1 FSHD.

14. The method according to claim 1, wherein the FSHD is Type 2 FSHD.

15. The method according to claim 1, wherein the patient is a mammal, non-human mammal, or a human.

16. The method according to claim 1, wherein the patient exhibits one or more symptoms of FSHD.

17. The method of claim 16, wherein the symptoms of FSHD are facial muscle weakness, shoulder weakness, hearing loss, abnormal heart rhythm, unequal weakening of muscles in the upper body, loss of strength in the abdominal muscles, or foot drop.

18. The method of claim 17, wherein the symptom of FSHD is facial muscle weakness.

19. The method of claim 17, wherein the unequal weakening of muscles in the upper body is unequal weakening of muscles in the biceps, triceps, deltoids, or lower arm muscles.

20. The method according to claim 1, wherein the method further comprises administering a second therapy for FSHD.

21. The method of claim 20, wherein the second therapy is administered before the inhibitor of p38.

22. The method of claim 20, wherein the second therapy is administered concurrently with the inhibitor of p38.

23. The method of claim 20, wherein the second therapy is administered after the inhibitor of p38.

24. The method according to claim 20, wherein the second therapy is a BET inhibitor.

25. The method of claim 24, wherein the BET inhibitor is I-BET762, I-BET726, I-BET151, RVX-208, CPI-203, CPI-232, CPI-0610, (+) JQ1, OTX-015, GW-841819X, BET-BAY-022, SRX-2523, or ABBV-075.

26. The method according to claim 20, wherein the second therapy is a β-2 adrenergic receptor agonist.

27. The method of claim 26, wherein the β-2 adrenergic receptor agonist is bitolterol, fenoterol, isoprenaline, levosalbutamol, orciprenaline, pirbuterol, procaterol, ritodrine, salbutamol, bambuterol, formoterol, arformoterol, clenbuterol, salmeterol, abediterol, indacaterol, or olodaterol.

28. The method according to claim 20, wherein the second therapy comprises the BET inhibitor and the β-2 adrenergic receptor agonist.

29. The method of claim 28, wherein the BET inhibitor is I-BET762, I-BET726, I-BET151, RVX-208, CPI-203, CPI-232, CPI-0610, (+) JQ1, OTX-015, GW-841819X, BET-BAY-022, SRX-2523, or ABBV-075 and the β-2 adrenergic receptor agonist is bitolterol, fenoterol, isoprenaline, levosalbutamol, orciprenaline, pirbuterol, procaterol, ritodrine, salbutamol, bambuterol, formoterol, arformoterol, clenbuterol, salmeterol, abediterol, indacaterol, or olodaterol.

30. The method of claim 20, wherein the second therapy is a therapy to increase muscle mass, physical therapy, or occupational therapy.

31. The method of claim 20, wherein the second therapy is a therapy which improves the quality of life.

32. The method of claim 31, wherein the second therapy is scapular fusion or scapular bracing.

33. The method of claim 31, wherein the second therapy is an anti-inflammatory compound.

34. The method of claim 33, wherein the anti-inflammatory compound is an NSAID.

35. The method of claim 33, wherein the anti-inflammatory compound is a glucocorticoid receptor modulator (glucocorticoid).

36. The method according to claim 1, wherein the compound is administered systemically.

37. The method of claim 36, wherein the compound is administered systemically via injection or oral administration.

38. The method according to claim 1, wherein the inhibitor of p38 is administered once.

39. The method according to claim 1, wherein the inhibitor of p38 is administered two or more times.

40. A pharmaceutical composition formulated for the treatment of facioscapulohumeral muscular dystrophy (FSHD) comprising a p38 inhibitor and an excipient.

41. The pharmaceutical composition of claim 40, wherein the pharmaceutical composition is formulated for administration orally or via injection.

42. The pharmaceutical composition of claim 41, wherein the pharmaceutical composition is formulated for administration via injection.

43. The pharmaceutical composition of claim 42, wherein the pharmaceutical composition is formulated for administration via intravenous, subcutaneous, or intramuscular injection.

44. The pharmaceutical composition according to claim 40, wherein the pharmaceutical composition is formulated as a unit dose.
